(19) United States
(12) Patent Application Publication
(10) Pub. No.: US 2011/0172724 A1
(43) Pub. Date: Jul. 14, 2011

(54) BIOCOMPATIBLE MAGNESIUM MATERIAL

(75) Inventors: Norbert Hort, Luneburg (DE); Hajo Dieringa, Sudergellersen (DE); Karl Ulrich Kainer, Hohnstorf (DE)

(73) Assignee: GKSS-FORSCHUNGSZENTRUM GEESTHACHT GMBH, Geesthacht (DE)

(21) Appl. No.: 12/097,461

(22) PCT Filed: Dec. 14, 2006
(86) PCT No.: PCT/EP2006/012050

§ 371 Date: Nov. 24, 2009

(30) Foreign Application Priority Data
Dec. 14, 2005 (DE) 10 2005 060 203.7

(51) Int. Cl. A61B 17/58; A61K 47/02; B02C 23/00

(52) U.S. Cl. 606/86 R; 106/286.6; 106/286.4; 106/286.5; 241/25

(57) ABSTRACT

A biocompatible material from which solid structures such as for example screws or plates can be manufactured, which are used for fixing bone fractures or damage and display an adequate mechanical stability. A mixture of apatite and a magnesium alloy, in the form of chips or powder, is ground in a ball mill until a homogeneous mixture forms. The homogeneous mixture is consolidated in a second step. This can be carried out by extrusion or forging. The desired shape can then be extracted from the obtained solid material by machining.

The present invention relates to a process for the preparation of a biocompatible material from which structures for fixing bone fractures or damage can be produced.

Bones represent a material which is subject to gradual change. This means that the properties, in particular the porosities, undergo constant localized changes. An abrupt change in the properties, which would lead to mechanical instability at the boundary surface (Corticalis Spongiosa), is avoided. An optimum bone replacement material should therefore imitate this graduated structure in order to provide the desired properties, such as mechanical stability, degree of degradation, porosity with local variation. On the other hand, bioresorbable or biodegradable implants which dissolve on their own after the damage has been repaired, thus enabling a second operation for explantation to be avoided, are desirable in the field of bone reconstruction. Such a biodegradable implant made of biodegradable metal is known from DE 197 31 021.

Such an implant material must display an adequate mechanical stability and the biodegradation must take place at a decomposition rate synchronized with the bone healing process. Bioresorbable polymer implants are used for example as alternatives to titanium. Currently the most important group of resorbable synthetic-organic materials comprises linear, aliphatic polyesters, in particular polylactides and polyglycolides based on lactic acid and glycolic acid. These materials retain their strength during the healing process and slowly decompose through hydrolysis into lactic acid. Due to their limited mechanical stability, however, they are preferably used for non-load-bearing bone segments.

In the field of synthetic, inorganic bone replacement materials, attempts are being made to provide skeletons, in particular made of ceramic bone replacement materials, into which the bone tissue can grow for bone regeneration. However, due to the brittleness of the mechanical materials, they cannot absorb substantial mechanical loads. So-called composite materials are used to increase the mechanical strength and load-bearing capacity of these skeletons made of ceramic materials.

Biodegradable metal implant materials such as magnesium alloys also offer a degree of mechanical stability and are therefore of increasing interest. Such implant materials are described in U.S. Pat. No. 3,687,135 and DE-A-102 53 634. However, these materials are not biocompatible, i.e. completely biologically compatible.

The object of the present invention is to provide a process for the production of a biocompatible material from which solid structures such as for example screws or plates can be manufactured, which are used for fixing bone fractures or damage and display an adequate mechanical stability. This object is achieved by a process in which firstly a mixture of apatite and a magnesium alloy in the form of chips or powder is ground in a ball mill until a homogeneous mixture forms. The homogeneous mixture is consolidated in a second step. This can be carried out by extrusion or forging. The desired shape can then be extracted from the obtained solid material by machining.

The object is also achieved by a biocompatible material, suitable for fixing bone fractures and damage, which contains a homogeneous mixture of apatite and a magnesium alloy.

The magnesium alloy preferably contains aluminium, particularly preferably in a quantity of 0 to 15 wt.-%, more preferably 1 to 10 wt.-%. It can also contain zinc, preferably in a quantity of 0 to 7 wt.-%, particularly preferably 1 to 5 wt.-%, tin, preferably in a quantity of 0 to 6 wt.-%, particularly preferably 1 to 4 wt.-%, lithium, preferably in a quantity of 0 to 5 wt.-%, particularly preferably 0.5 to 4 wt.-%, manganese, preferably in a quantity of 0 to 5 wt.-%, particularly preferably 1 to 4 wt.-%, silicon, preferably in a quantity of 0 to 5 wt.-%, particularly preferably 1 to 4 wt.-%, calcium, preferably in a quantity of 0 to 3 wt.-%, particularly preferably in a quantity of 1 to 3 wt.-%, yttrium, preferably in a quantity of 0 to 5 wt.-%, particularly preferably in a quantity of 0.5 to 4 wt.-%, strontium, preferably in a quantity of 0 to 4 wt.-%, particularly preferably 0.1 to 3 wt.-%, one or more metals, selected from the group of the rare earths, preferably in a quantity of 0 to 5 wt.-%, particularly preferably in a quantity of 0.1 to 3 wt.-%, silver, preferably in a quantity of 0 to 2 wt.-%, particularly preferably 0.1 to 2 wt.-%, iron, preferably in a quantity of 0 to 0.1 wt.-%, nickel, preferably in a quantity of 0 to 0.1 wt.-% and/or copper, preferably in a quantity of 0 to 0.1 wt.-%.

The preferred weight ratio of apatite to magnesium alloy is 100:1 to 1:100, more preferably 20:1 to 1:20 and in particular 1:5 to 5:1.

It was found that a structure, strengthened compared with the matrix alloy, comprising alloy and apatite particles is obtained, in which the non-metal apatite particles are finely dispersed in the metal matrix. Implants made of this material offer above all a higher mechanical stability compared with the known biodegradable implants. The magnesium alloy is gradually corroded. The finely distributed apatite portions are thus released over a prolonged period and support the body tissue during healing and bone growth. Because strength also plays an important part, in addition to the described properties, a strengthening of the dispersion is also achieved in this material by the finely distributed non-metallic constituents in the metal matrix. This means that the material is significantly strengthened compared with the matrix alloy. Screws and plates which are made of this material display an increase in strength compared with unreinforced magnesium alloys which, as corroding materials, could also be used as implants without an apatite portion.

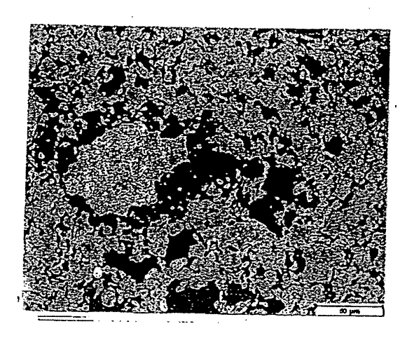

FIG. 1 is a light-microscope image of the microstructure of the material. The dark area is the intercalated apatite. The light area is the magnesium matrix. It can be seen that the apatite is dispersed homogeneously in the magnesium matrix.

1. Material for fixing bone fractures and/or damage which contains a homogeneous mixture of apatite and a magnesium alloy.

2. Material according to claim 1, characterized in that the magnesium alloy contains aluminium.

3. Material according to claim 2, characterized in that the aluminium content of the magnesium alloy is 1 to 15 wt.-%.

4. Material according to claim 1, characterized in that the magnesium alloy contains zinc.

5. Material according to claim 4, characterized in that the zinc content of the magnesium alloy is 1 to 7 wt.-%.

6. Material according to claim 1, characterized in that the magnesium alloy contains tin.

7. Material according to claim 6, characterized in that the tin content of the magnesium alloy is 1 to 6 wt.-%.

8. Material according to claim 1, characterized in that the magnesium alloy contains zinc.

9. Material according to claim 8, characterized in that the zinc content of the magnesium alloy is 0 to 7 wt.-%.

10. Material according to claim 1, characterized in that the magnesium alloy contains lithium.

11. Material according to claim 10, characterized in that the lithium content of the magnesium alloy is 1 to 5 wt.-%.

12. Material according to claim 1, characterized in that the magnesium alloy contains manganese.

13. Material according to claim 12, characterized in that the manganese content of the magnesium alloy is 1 to 5 wt.-%.

14. Material according to claim 1, characterized in that the magnesium alloy contains yttrium.

15. Material according to claim 14, characterized in that the yttrium content of the magnesium alloy is 1 to 5 wt.-%.

16. Material according to claim 1, characterized in that the magnesium alloy contains a metal selected from the group consisting of the rare earths.

17. Material according to claim 16, characterized in that the rare earths content of the magnesium alloy is 1 to 5 wt.-%.

18. Material according to claim 1 characterized in that the weight ratio of apatite to magnesium alloy is 1:100 to 100:1.

19. Material according to claim 18, characterized in that the weight ratio of apatite to magnesium alloy is 1:20 to 20:1.

20. Material according to claim 18, characterized in that the weight ratio of apatite to magnesium alloy is 1:5 to 5:1.

21. Process for the production of a biocompatible material for fixing bone fractures and/or damage comprising:
grinding a mixture of apatite and a magnesium alloy until a homogeneous mixture forms, and then consolidating the homogeneous mixture into a structure.

22. The process of claim 21 wherein the mixture is consolidated into a shape selected from the group consisting of a screw, a plate, and an implant.

23. The process of claim 21, wherein the mixture is ground in a ball mill.

24. A method of fixing bone fractures and/or damage comprising connecting the bone to the material of claim 1.
